(19) United States
(12) Patent Application Publication
(10) Pub. No.: US 2009/0246139 A1
(43) Pub. Date: Oct. 1, 2009

(54) Efficient methods to isolate effectors of proteins involved in olfactory or chemosensory pathways and efficient methods to use these effectors to alter organism olfaction, chemosensation, or behavior

(76) Inventors: Daniel F. Woods, Irvine, CA (US); Spiros D. Dimitratos, Irvine, CA (US)

Correspondence Address: DAVID R PRESTON & ASSOCIATES APC, 5850 OBERLIN DRIVE, SUITE 300, SAN DIEGO, CA, 92121, US

(21) Appl. No.: 12/387,009

(22) Filed: Apr. 27, 2009

Related U.S. Application Data
(62) Division of application No. 11/372,777, filed on Mar. 10, 2006, now abandoned.
Application No. 11/372,777 is a continuation-in-part of application No. 10/106,749, filed on Mar. 26, 2002, now abandoned.
(60) Provisional application No. 60/279,168, filed on Mar. 27, 2001.

(30) Foreign Application Priority Data
Mar. 26, 2002 (US) PCT/US02/09559

(51) Int. Cl. A61K 49/00; A61K 38/16; A01N 61/00; A01P 7/04; A01P 17/00

(52) U.S. Cl. 424/9.2; 514/12; 424/84

(57) ABSTRACT

This invention provides methods and compositions for identifying effectors, binding partners, or other molecules that interact with the proteins involved in the chemosensory pathway; examples of proteins involved in the olfactory pathway include odorant binding proteins (OBPs), sensory appendage proteins (SAPs), orthologs of the Drosophila melanogaster Takeout protein (TOLs), odorant degrading enzymes (ODEs) and odorant receptors (ORs or GPCRs). The invention identifies proteins, molecules, or chemicals that can interact with these olfactory proteins, including but not limited to agonists or antagonists of these proteins. This invention also provides methods and compositions for identifying effectors, binding partners, or other molecules that interact with the proteins involved in the chemosensory pathway; these proteins are generally similar to the olfactory proteins. Generally, the method consists of isolating gene products specifically expressed in the tissue of interest, and assaying function. This invention provides methods of use for the identified agonists and antagonists for controlling insect feeding and breeding behavior, eliminating odors, altering other behaviors, and the like.

This application is a divisional of U.S. patent application Ser. No. 11/372,777, filed Mar. 10, 2006, entitled “Efficient Methods to Isolate Effectors of Proteins Involved in Olfactory or Chemosensory Pathways and Efficient Methods to use these Effectors to Alter Organism Olfaction, Chemosensation, or Behavior” which is incorporated herein by reference in its entirety, which claims benefit of priority of U.S. patent application Ser. No. 10/106,749, filed Mar. 26, 2002, entitled “Efficient methods for isolating functional G-protein coupled receptors and identifying active effectors and efficient methods to isolate proteins involved in olfaction and efficient methods to isolate and identify active effectors” which is incorporated herein by reference in its entirety, which claims benefit of priority of Provisional U.S. patent application Ser. No. 60/279,168, filed Mar. 27, 2001, entitled “Efficient methods for isolating functional G-protein coupled receptors and identifying active effectors,” which is incorporated herein by reference in its entirety. This application also claims benefit of priority of Provisional U.S. Patent Application Ser. No. 60/353,392, filed Jan. 31, 2002, entitled “Efficient methods for isolating functional G-protein coupled receptors and identifying active effectors and efficient methods to isolate proteins involved in olfaction and identify active effectors or interactors,” which is incorporated herein by reference in its entirety.

### TECHNICAL FIELD

The present invention generally relates to methods and compositions for identifying, isolating and utilizing chemosensory or neuronal proteins from any species where these proteins are expressed. The invention also relates to methods for isolating either natural or synthesized proteins or chemicals that interact with chemosensory proteins or other neuronal proteins. Methods facilitating the in vivo evaluation of synthesized proteins or chemicals for interaction with chemosensory proteins are also provided. The technologies presented herein are feasible in a broad range of applications including in the control of insect species, whether these species are considered pests, beneficial, or neutral, via behavior alteration.

### BACKGROUND

Odor detection, olfaction, taste, gustation, and chemosensation have been studied extensively in vertebrates and invertebrates alike, yet the molecular mechanisms responsible for these processes have not been entirely elucidated; many aspects of the chemosensory process remain unknown.¹ Interestingly, odors, scents and tastes control many crucial aspects of insect behavior, including mating and feeding.^{1,2} Subsequently, insects have evolved extremely sensitive chemosensory systems. Insects can detect exceedingly faint odors and distinguish one odor from another extremely well. Odor detection in insects is effected by an extensive signaling cascade that affords the process such high efficiency and specificity. This cascade is localized in the antennae of most species.^{1,3}

Although details regarding the mechanics of insect olfaction and the identity of the molecules involved remain unknown, it is known that odorant binding proteins are responsible for binding lipophilic or hydrophobic scents such as sex pheromones and guiding them across the hydrophilic extracellular matrix of the antennal tissue. Researchers have speculated that these odorant binding proteins or OBPs are necessary to allow hybrophobic molecules such as most scents or odors to transverse the hydrophilic extracellular matrix and reach the surface of neuronal cells in the antennae. These neuronal cells express odorant receptors, members of the large G-protein-coupled receptor family, that bind specific odors or pheromones and initiate an elaborate intracellular signaling cascade that results in odor detection. The mechanisms and classes of molecules responsible for invertebrate gustation are the same as those involved in olfaction.¹

Since odorant molecules are often present in the atmosphere in only minute amounts, they are difficult to analyze or even isolate in adequate quantities for analysis to be feasible. Yet odorant molecules control many aspects of insect behavior,^{1,4-8} and harnessing their power to control insect pest species is particularly attractive since odors and tastes, unlike pesticides, are non-toxic. Furthermore, their effects are usually species-specific, meaning they are highly targeted—again, in contrast to conventional insecticides. There is therefore a need for a more thorough understanding of the nature of those odors, semiochemicals, and pheromones capable of drastically or usefully altering insect pest behavior. There is also a need to understand insect chemosensation better, particularly at the molecular level.

The invention provides means and methods to rapidly identify and characterize chemosensory proteins (such as odorant binding proteins) from insects or other species, their agonists, and their antagonists, leading to the development of a number of pest control or odor control products. OBPs can be used to concentrate an odor, prevent an odor from being detected, or affect the length of time an odor is detected (generally referred to as the odor's active life or “half life”). OBPs are relatively abundant proteins that are specifically expressed in insect antennal tissue. The present invention recognizes the need to rapidly isolate agonists and antagonists of OBPs, and provides the methods necessary to do so. Other classes of chemosensory proteins that the present invention provides means and methods to rapidly identify agonists and antagonists for include sensory appendage proteins (SAPs), odorant degrading enzymes (ODEs), orthologs of the Drosophila melanogaster Takeout protein (TOLs, for Takeout-likes), odorant receptors (ORs), gustatory receptors (GRs) and other proteins involved in olfaction, gustation, chemosensation, behavior, or the regulation of circadian rhythms.^{1,3,9-11}

### BRIEF DESCRIPTION OF THE FIGURES

FIG. 1. A model of novel repellent function based on inducing anosmia. Our novel mosquito repellents are based on molecules identified from screening combinatorial chemical libraries. These molecules are selected for their ability to bind chemosensory proteins and render them unable to interact correctly with other molecular effectors of the chemosensory (olfactory, gustatory) pathway. In this example we examine the induction of anosmia as a result of targeting an OBP. (a) Hydrophobic odorants enter the haemolymph of mosquito antennal tissue and are bound by OBPs that transport them through the hydrophilic medium to the surface of olfactory neurons, where the OBPs are bond by ORs. This initiates the olfactory signaling cascade and results in behavioral response from the mosquito.⁹ (b) In the presence of an OBP-binding molecule, OBPs cannot bind natural odorants and the olfactory or gustatory signaling cascade is blocked, thus, repellents based on OBP-binding molecules induce anosmia. This same general mechanism would also hold true for other chemosensory proteins.

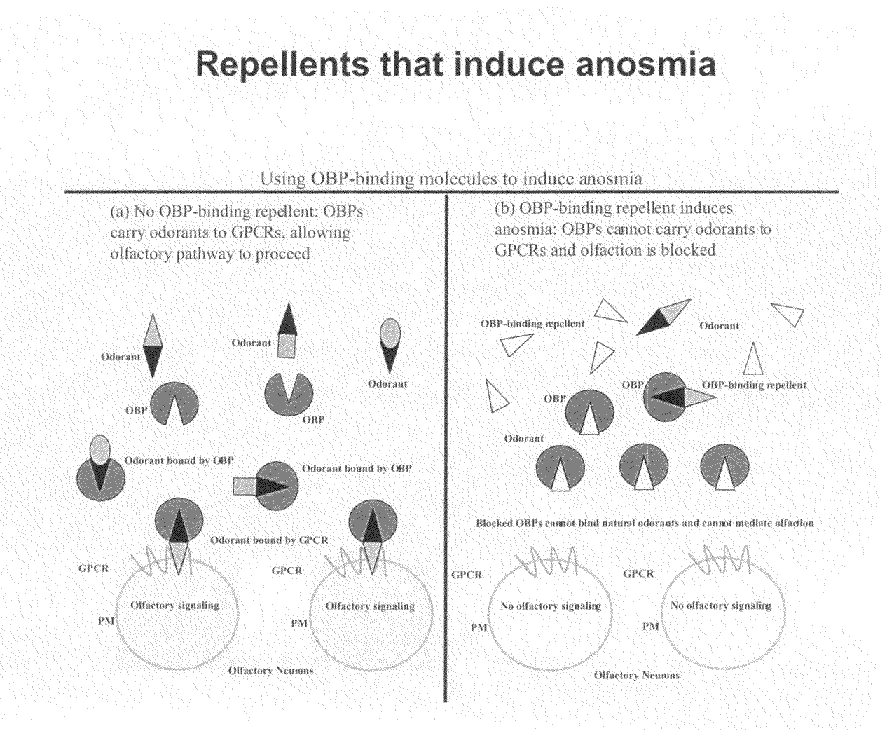

### SUMMARY

The present invention recognizes the need to rapidly and reliably identify novel potential interactors for chemosensory proteins or other proteins that control the manner in which organisms recognize and/or respond to olfactory, gustatory, or other chemical cues in the environment. The present invention therefore permits the identification of novel chemosensory protein interactors based on screening combinatorial chemical libraries using rational design.

Possible applications of the invention include but are not limited to the development of novel, species-specific pesticide or insecticide alternatives that are compliant with the Food Quality Protection Act (FQPA) and operate based on mating disruption or the alteration of other scent-controlled behaviors in arthropod pests, and the development of pest monitoring systems that operate based on the presence of pest pheromone in situ. Furthermore, the invention can be used to isolate a host of novel semiochemicals with desirable effects on specific species, e.g. the induction of anosmia or the effective masking of odors. The invention can thus be applied to the development of methods or devices that induce anosmia in a variety of species.

### DETAILED DESCRIPTION OF THE INVENTION

### Definitions

Unless otherwise stated, all scientific and technical terms used herein have the same meaning as commonly understood by one of ordinary skill in the art to which this invention belongs. Generally, the nomenclature used herein and the laboratory procedures in molecular biology, molecular genetics, biochemistry, physical chemistry, cell culture, protein chemistry, and nucleic acid chemistry described below are those well known and commonly employed in the art. Standard techniques are used for recombinant nucleic acid methods, eukaryotic transformation, and microbial culture and transformation. Enzymatic reactions and purification steps are performed according to the manufacturer's instructions unless otherwise noted. Techniques and procedures are generally performed according to conventional methods in the art. General references include Sambrook et al., Molecular Cloning: A Laboratory Manual, 2^{nd} Ed. (1989) Cold Spring Harbor Laboratory Press, Cold Spring Harbor, N.Y., USA, and Ashburner, M., Drosophila: A Laboratory Manual (1989) Cold Spring Harbor Laboratory Press, Cold Spring Harbor, N.Y., USA. The laboratory procedures described in combinatorial chemistry, synthetic chemistry, and electrophysiology, and the nomenclature used are those well known and commonly employed in the art. As employed throughout the disclosure, the following terms, unless otherwise indicated, shall be understood to have the following meanings:

“Agonist” refers to a molecule that binds a protein such as a chemosensory protein and causes its activation, leading to a signal being transduced or converted that elicits a certain behavioral response (e.g. a pheromone molecule that induces mating behavior).

“Agustia” refers to the inability to detect a taste.

“Allomone” refers to a compound produced by a member of one species that affects the behavior of a member of another species.

“Anosmia” refers to the inability to detect a scent, smell, or odor.

“Antagonist” refers to a molecule that binds a protein such as a chemosensory protein and blocks its activation by an agonist, (e.g. a molecule that inhibits mating behavior).

“Arometics” refers to small synthetic molecules isolated from the combinatorial chemical libraries that will act as either agonists or antagonists to the targeted chemosensory protein(s). Although they can bind the same chemosensory proteins as native pheromones, Arometics are not the native pheromone. The term “Pheromone mimetics” is also used to describe these molecules.

“Bioinformatics” refers to the discipline that integrates biotechnology and modem computational, statistical, and analytical or mathematical methods.

“cDNA” refers to complementary DNA, which is a DNA copy of the mRNA or messenger RNA expressed in the cell. The term “cDNA” therefore represents gene products or transcripts.

“Chemosensory protein” refers to a protein component of the chemosensory system including the olfactory and gustatory system. Chemosensory proteins can be soluble, insoluble, membrane-bound, extracellular, secreted, or intracellular

“Codlemone” refers to pheromone of the codling moth, Cydia pomonella Linnaeus.

“Combinatorial chemical libraries” refers to large, randomly constructed libraries of small molecules; these libraries will be used in screening for potential substitutes to naturally occurring pheromones.

“Domain” refers to an area of a protein with a specific function or exhibiting a specific structural motif.

“Effectors” refers to naturally occurring or synthetic molecules, or compounds capable of interacting with a chemosensory protein under study. Effectors can be agonists or antagonists.

“Electroantennogram” refers to the output of a device incorporating electrodes that measure electrical activity across an antenna mounted in conductive medium (typically a gel). In this manner, the response of receptors on the antenna to stimuli including odors can be quantified.

“FQPA” refers to the Food Quality Protection Act of 1996 that requires all present tolerances for pesticides to undergo risk assessments under more stringent standards; the implications of this legislation suggest that several widely used organophosphates and carbamates will be slowly phased.

“Genomics” refers to the cloning and molecular characterization of entire genomes.

“Genetically Modified Organism (GMO)” refers to an organism that has been genetically engineered using gene splicing or molecular biology techniques (vs. a traditional breeding approach) to exhibit specific genetic traits.

“G-protein coupled receptors (GPCRs)” refers to pheromone or odorant serpentine receptors that bind trimeric G-proteins within the cell. Also called odorant receptors (ORs).

“High throughput bioassay” refers to an assay system based on a biological response that can be accomplished on a very large scale (>1000/day).

“High throughput sequencing” refers to a DNA sequencing system that allows for the determination of a very large number of nucleotides (>32,000 base pairs/day).

“Homologs” refers to genes that have a common ancestry. Homologs are divided into orthologs, that are homologs with the same function as the ancestral gene, and paralogs, that are homologs with a different function from the ancestral gene.

“Homology” refers to the extent of similarity between the DNA sequences encoding two or more genes, or the amino acid sequences comprising two or more proteins, as in a gene or protein family.

“Hybridization” refers to selective and specific binding, typically between a probe and its target.

“Hydrophobicity” refers to the solubility of a particular protein in water.

“Kairomone” refers to a compound that is an interspecific chemical message that benefits the receiving species.

“Known pheromone” refers to a pheromone already identified that mediates a specific behavioral response.

“Lead Chemical” refers to a chemical suspected to have the ability to interact with a chemosensory protein, thus making it a candidate pheromone mimetic or arometic.

“Mating disruption” refers to a method of pest control most commonly found in agriculture; it involves saturating the crop environment with a sex pheromone in order to confuse the males and prevent them from locating females.

“mRNA” refers to that portion of RNA comprising sequences that are translated into proteins. Only a portion of the RNA present in a cell is mRNA; other RNA forms include ribosomal RNA and transfer RNA. Only mRNA encodes proteins.

“Odorant Binding Proteins” or “OBPs” refers to proteins in sensory tissues believed to bind odors, that are typically hydrophobic, and escort them across the hydrophilic extracellular matrix to the cell surface, where odorant receptors are located.

“Odorant Degrading Enzyme” or ODE refers to a diverse group of enzymes involved in the re-potentiation of the chemosensory system. ODEs are responsible for degrading stimuli from the environment after they are sensed by the organism.

“Odorant Receptor” or OR refers to the subcellular structures located in the plasma membrane of insect neuronal cells that are responsible for initiating the organism's perception of a specific odor—that is, they allow the organism to smell various scents and odors. Also called a GPCR.

“Odorant” refers to smell, scent, or odor.

“PCR” refers to the Polymerase Chain Reaction, a method of amplifying nucleic acid sequences in vitro in order to obtain larger amounts of DNA.

“Pheromone Mimetics” refers to small synthetic molecules isolated from the combinatorial chemical libraries that will act as either agonists or antagonists to the targeted pheromone receptors. Although they can bind the same receptors as native pheromones, Pheromone Mimetics are not the native pheromone. The term “Arometics” is also used to describe these molecules.

“Pheromone” refers to an odorant chemical released by an insect that causes a specific interaction with another insect of the same species.

“Probe” refers to a labeled DNA fragment, RNA fragment, protein fragment, or chemical that can hybridize to a specific region of a target DNA or protein segment, and whose presence can be readily assayed.

“Promotor” refers to a segment of DNA that controls gene expression in vivo, capable of limiting expression spatially and/or temporally.

“Reagents” refers to chemicals and compounds (either naturally occurring or synthetic) or enzymes used in a chemical reaction to measure or yield other substances.

“Reporter Gene” refers to a gene used in biological or biochemical experiments in order to monitor an interaction. Reported genes respond to protein-protein interactions by triggering an effect that is easily detectable, e.g. the emission of fluorescent light or the production of an assayable product.

“Semiochemicals” refers to chemicals (scents, odors, tastes, pheromones, pheromone-like compounds, or other chemosensory compounds) that mediate interactions between organisms.

“Sensory Appendage Proteins” or SAPs refers to soluble secreted proteins present in the hemolymph of chemosensory organs, and to the orthologs of these proteins. SAPs are thought to complex with odorant or gustatory molecules and escort them to neuronal cell surfaces. These proteins usually feature four cysteine residues.

“Serpentine receptors” refers to GPCRs or ORs; this term is based on the actual structure of the protein in the cell membrane (seven transmembrane passes in a serpentine shape).

“Signal transduction cascade” refers to a series of molecules in a cell that transduces or converts an external signal (e.g. a pheromone) into a downstream response within the cell (e.g. a change in gene activity).

“Synomone” refers to a compound produced by one organism that affects the behavior of an organism of another species; both organisms benefit.

“Takeout” or TO refers to a protein encoded by the takeout gene in Drosophila melanogaster. TO is involved in the regulation of circadian rhythms and thought regulate feeding and mating behaviors. TO-like or TOL proteins are orthologs of Drosophila Takeout.

“Trans-gene” refers to a gene that has been introduced into the genome of a cell or organism by transformation.

“Transmembrane Domains” refers to hydrophobic domains of a protein that penetrate the cell membrane.

“Unknown pheromone” refers to a pheromone not yet determined or identified that mediates a specific behavioral response.

### Introduction

The present invention recognizes the need to identify novel chemosensory proteins and their interactors in either a cell based or cell-free system. Since the identification of novel chemosensory proteins and their interactors can be performed in living cells, cell lines can be developed to allow further functional characterization and/or the isolation of other interacting proteins or effectors, regardless of effector origin (synthetic or naturally occurring substance). The invention thus provides distinct advantages over existing methods used to isolate, identify, and characterize novel protein family members and their interactors.

What follows is a non-limiting introduction to the breadth of the invention, including several general and useful aspects:

1) The invention provides a method of identifying genes encoding novel chemosensory proteins, including odorant binding proteins, sensory appendage proteins, odorant degrading enzymes, the homologs of these three protein classes involved in gustation or other chemical senses, orthologs of the Drosophila melanogaster Takeout protein, and other proteins involved in chemosensation, behavior, or the regulation of circadian rhythms.

2) The invention provides a method for identifying molecules, chemicals, or reagents, either synthetic or occurring in nature, that interact with isolated chemosensory proteins without prerequisite knowledge of the native ligand's structure. Identification is accomplished using a cell-based system that allows high-throughput assays or a cell-free system that also allows high throughput. These methods can therefore employ large combinatorial chemistry libraries to identify receptor ligands, whether the ligands are agonists, antagonists or have a novel function. These ligands may mimic the function of native pheromones, kairomones, synomones, or allomones and are therefore called Arometics or “Pheromone Mimetics.”

3) The invention provides a method to assay the activity of potential Arometics using transformed Drosophila melanogaster, either as a transformed whole organism, transformed dissected sensory organs, or cultured transformed cell lines. For example, a novel chemosensory protein can be transformed into Drosophila using tools readily available in the art, and then assayed in any of three different manners for interaction with a lead compound identified from assaying a combinatorial chemical library (as described above):
(a) The entire transformed organism can be exposed to the lead chemical and assayed for a behavioral response.
(b) The antennae from transformed Drosophila can be dissected and assayed for a response to the lead chemical using an electroantennogram¹² or similar method.
(c) The transformed organisms can be used to develop stable cell lines that can be cultured in vitro, and these cell lines can be assayed for a response to the lead chemical via a variety of methods, including but not limited to coupling the chemosensory protein to a reporter gene cascade.

4) The invention provides methods, technologies, and compositions necessary to induce anosmia in a variety of species, ranging from arthropods to humans. Applications range from pest control to odor masking in agricultural, commercial, and domestic environments.

5) The invention provides methods and compositions that can be utilized in the development of repellants or attractants useful in the control or behavioral manipulation of a wide variety of invertebrate and vertebrate species. The species this invention can be applied to include but are not limited to:
(a) Invertebrates: dipterans (e.g. mosquitoes, gnats, flies), termites, lepidopterans (e.g. moths, butterflies), orthopterans (e.g. grasshoppers and locusts), sharpshooters (e.g. Homalodisca spp.), cockroaches, beetles, ants, fleas, silverfish, hymenopterans (e.g. wasps, bees, hornets), kissing bugs (e.g. Triatoma dimidiatamyria), other insects, myriapods (e.g. millipedes and centipedes), mites, spiders, ticks, other arachnids, terrestrial isopods (e.g. pill bugs and sow bugs), other arthropods, annelids, nematodes, mollusks (e.g. snails and slugs).

Vertebrates: rodents, lagomorphs, insectivora (e.g. moles and shrews), chiroptera, carnivora (e.g. weasels, coyotes, bears, dogs, and cats), artiodactyls, perissodactyls, primates (including humans), other mammals, reptiles, marine vertebrates including agnatha, chondrichthyes (e.g. sharks) and osteichthyes, aves (e.g. pigeons).

### I. METHODS TO DEVELOP DEVICES THAT REDUCE A TARGET SPECIES' SENSITIVITY TO ODORS, TASTES, OR OTHER STIMULI DETECTABLE BY THE SPECIES' CHEMOSENSORY SYSTEM

Mosquitoes such as Anopheles gambiae use olfactory or other chemosensory stimuli as a means of identifying potential blood meal hosts.^{1,3,9,13,14} Consequently, devices that reduce the mosquitoes' sensitivity to odors can control pests in an environmentally responsible manner. The present invention recognizes this need and provides means, methods, and constructs to develop devices capable of reducing mosquito sensitivity to the odors commonly used to locate human hosts. The goal of these devices is to induce anosmia in as many species of mosquito as possible. Since chemosensory proteins and proteins that control important aspects of the insect olfactory and gustatory systems appear to be conserved across species, such a goal is realistic.

For example, this method can take advantage of the interspecific conservation of OBPs.¹⁵ OBPs from several target species can be isolated as described in this Application. Once cloned, DNA sequences encoding these OBPs can be inserted into expression vectors so that OBPs can be expressed in vitro, using tools common in the art that include transgenic prokaryotic cells, eukaryotic cell lines or transgenic animals. Combinatorial chemical libraries are screened for compounds capable of interacting with the OBPs in vitro or in vivo, and these compounds are then incorporated into products capable of altering pest species' behavior based on their scent.

These methods can also be employed to reduce a species' behavioral response or detection capability to a stimulus by targeting other chemosensory proteins instead of or in addition to OBPs;¹ such proteins include sensory appendage proteins (SAPs),¹ odorant degrading enzymes (ODEs),¹ orthologs of the Drosophila melanogaster Takeout protein (TOLs, for Takeout-likes),^{3,9-11,16} odorant receptors (ORs),¹ gustatory receptors (GRs),¹⁷⁻¹⁹ pheromone binding proteins, circadian rhythm proteins and other proteins involved in olfaction, gustation, chemosensation, the sensory system, or the regulation of chemosensory-mediated behavior.^{1,4,5,7,8} Molecules or compounds identified in this manner as interacting with a chemosensory protein of interest are called Arometics and can subsequently be evaluated for behavioral effects on living organisms. The sensitivity of an organism's response to or detection of a stimulus can be reduced in the manner described here to the point of anosmia or agustia.

### II. METHODS TO IDENTIFY SMALL SYNTHETIC MOLECULES OR COMPOUNDS THAT BIND CHEMOSENSORY PROTEINS VIA SURFACE PLASMON RESONANCE

The invention recognizes the need to isolate agonists or antagonists of chemosensory proteins from a variety of organisms. These chemosensory proteins include but are not limited to odorant binding proteins, gustatory binding proteins, sensory appendage proteins, circadian rhythm proteins, orthologs of these listed proteins, and orthologs of behavioral proteins such as the Takeout protein from Drosophila melanogaster.^{1,9,11,16,20}

The chemosensory protein controlling a specific behavior like mating or feeding is identified using one of the methods common in the art, including but not limited to bioinformatic analysis of DNA sequences, amino acid sequences, or protein tertiary structure. Once the chemosensory protein is identified, it is expressed in vitro in the form of a recombinant fusion construct or using other commonly available expression systems, and generated in quantities sufficient for subsequent experimental analysis. The recombinant protein can then be exposed to large collections of potential binding partners, including other proteins, synthetic molecules, naturally occurring molecules, and other compounds such as can be found in combinatorial and/or natural chemical libraries. The chemosensory protein is immobilized on a surface, covered with a buffer solution or other solution, and exposed to a variety of molecules or compounds flowing across this surface. Any interaction will result in a change in the total mass of compounds on the surface, and this change in mass is measured by surface plasmon resonance. This is the Deligo assay system.

Small molecules or compounds identified in this manner as interacting with a chemosensory protein of interest are called Arometics and can subsequently be evaluated for behavioral effects on living organisms.

### III. METHODS TO IDENTIFY SMALL SYNTHETIC MOLECULES OR COMPOUNDS THAT BIND CHEMOSENSORY PROTEINS VIA FLOW CYTOMETRY

The invention recognizes the need to isolate agonists or antagonists of chemosensory proteins from a variety of organisms. These chemosensory proteins include but are not limited to odorant receptors, gustatory receptors, odorant binding proteins, gustatory binding proteins, sensory appendage proteins, circadian rhythm proteins, odorant and gustatory degrading enzymes, orthologs of these listed proteins, and orthologs of behavioral proteins such as the Takeout protein from Drosophila melanogaster.^{1,9,11,16,20}

The chemosensory protein controlling a specific behavior like mating or feeding is identified using one of the methods common in the art, including but not limited to bioinformatic analysis of DNA sequences, amino acid sequences, or protein tertiary structure. Once the chemosensory protein is identified, it is expressed in vitro in the form of a recombinant fusion construct or using other commonly available expression systems, and generated in quantities sufficient for subsequent experimental analysis.

A number of dyes existing in the art, including N-phenyl-1-naphthylamine (1-NPN),²¹⁻²³ fluoresce when bound to the ligand-binding pocket of chemosensory proteins. The invention provides methods to take advantage of this property in order to identify molecular interactions between chemosensory proteins and other compounds. A given chemosensory protein is first bound to a solid support, such as a very small plastic bead, then allowed to bind a dye such as 1-NPN, resulting in fluorescence. This bound protein: 1-NPN compound is then exposed to a series of molecules including other proteins, synthetic molecules, naturally occurring molecules, and other compounds such as can be found in combinatorial and/or natural chemical libraries. Flow cytometry, as used commonly in the art, is used to detect the fluorescence quenching that results when the dye bound to the chemosensory protein's ligand-binding pocket is displaced by another molecule from the list of tested molecules or compounds. Fluorescence quenching thus indicates a molecular interaction. Binding different chemosensory proteins to various sized beads enables the multiplexing of this assay. Fluorescent quenching caused by binding of the molecule being tested can be assigned to a specific chemosensory protein based on the size of the particle (i.e. the bead).

Small molecules or compounds identified in this manner as interacting with a chemosensory protein of interest are called Arometics and can subsequently be evaluated for behavioral effects on living organisms.

### IV. METHODS TO IDENTIFY SMALL SYNTHETIC MOLECULES OR COMPOUNDS THAT BIND CHEMOSENSORY PROTEINS VIA MULTIPLEXED VARIABLE-WAVELENGTH SPECTROFLUOROMETRY

The invention recognizes the need to isolate binding partners, agonists or antagonists of chemosensory proteins from a variety of organisms. These chemosensory proteins include but are not limited to odorant binding proteins, gustatory binding proteins, sensory appendage proteins, circadian rhythm proteins, odorant and gustatory degrading enzymes, orthologs of these listed proteins, and orthologs of behavioral proteins such as the Takeout protein from Drosophila melanogaster.^{1,9,11,16,20}

The chemosensory protein controlling a specific behavior like mating or feeding is identified using one of the methods common in the art, including but not limited to bioinformatic analysis of DNA sequences, amino acid sequences, or protein tertiary structure. Once the chemosensory protein is identified, it is expressed in vitro in the form of a recombinant fusion construct or using other commonly available expression systems, and generated in quantities sufficient for subsequent experimental analysis.

A number of dyes existing in the art, including 1-NPN, fluoresce when bound to the ligand-binding pocket of chemosensory proteins. The invention provides methods to take advantage of this property in order to identify molecular interactions between chemosensory proteins and other compounds. The excitation and emission wavelengths of such dyes and the compounds the dyes form with a variety of chemosensory proteins will vary. The invention provides methods to use such dyes and a tuneable spectrofluorometer capable of reading multiple wells in a standard multiwell plate simultaneously in order to isolate molecular interactors of chemosensory proteins.

A given chemosensory protein is allowed to bind a dye such as 1-NPN,²¹⁻²³ resulting in detectable fluorescence. This protein: 1-NPN complex is then aliquoted into the wells of a multiwell plate as is common in the art. The contents of each well are then exposed to a molecule or pool of molecules including other proteins, synthetic molecules, naturally occurring molecules, and other compounds such as can be found in combinatorial and/or natural chemical libraries. A tuneable, variable-wavelength spectrofluorometer commonly available in the art is then used to identify those wells where the fluorescence resulting when the dye binds the chemosensory protein is quenched when the dye is displaced by another molecule or compound. This method of detecting intermolecular interactions and binding events is called the Attenu assay system.

Small molecules or compounds identified in this manner as interacting with a chemosensory protein of interest are called Arometics and can subsequently be evaluated for behavioral effects on living organisms.

### V. METHODS TO IDENTIFY SMALL MOLECULES THAT ARE CHEMOSENSORY PROTEIN AGONISTS OR ANTAGONISTS USING A CELL-BASED ASSAY

The invention recognizes the need to isolate agonists or antagonists of chemosensory proteins from a variety of organisms. Methods common in the art allow the screening of odorant receptors, gustatory receptors, and other receptors that are G-protein coupled receptors (GPCRs; also known as serpentine receptors and seven-transmembrane receptors).¹ The invention improves existing technologies by providing methods to enhance screening techniques with the use of odorant binding proteins, gustatory binding proteins, sensory appendage proteins, or other soluble chemosensory proteins that interact with chemicals or stimuli from the environment in vivo and form a complex with them.¹ These complexes are thought to enhance the sensitivity of the GPCR-based system in vivo,²⁴ and the invention provides for their use in vitro in order to enhance the sensitivity of a cell-based screening system.

A GPCR is selected for testing and inserted into a cell-based screening system in vitro as is common in the art. The odorant binding protein, gustatory binding protein, sensory appendage protein, or other soluble chemosensory protein present in cells normally expressing the GPCR in vivo is also inserted into the cell-based screening assay in order to enhance the assay's sensitivity by providing the soluble component of the in vivo system that binds a chemical stimulus, complexes with it, and escorts it to the membrane surface where the compound interacts with, and subsequently activates, the given GPCR.

This cell-based assay system is used to screen synthetic molecules, naturally occurring molecules, and other compounds such as can be found in combinatorial and/or natural chemical libraries for the ability to interact with the GPCR:soluble chemosensory protein pair in use. Small molecules or compounds identified in this manner as interacting with a chemosensory protein of interest are called Arometics and can subsequently be evaluated for behavioral effects on living organisms. These Arometics can alter an organism's behavior by manipulating the chemosensory system. For example, they can activate or block a chemosensory pathway eliciting a specific behavior such as attraction or repulsion, or can cause an organism to be unable to detect a specific scent, odor, or chemical, resulting in anosmia or agustia.

### VI. METHODS TO IDENTIFY SMALL SYNTHETIC MOLECULES THAT ARE CHEMOSENSORY PROTEIN AGONISTS OR CHEMOSENSORY PROTEIN ANTAGONISTS: “AROMETICS”

The invention provides a method to identify small molecules that mimic the effect of natural odors or scents, including natural pheromones, kairomones, allomones, and odors used by any pest species to identify their potential mates, food sources, or other aspects of their environment.¹ “Arometics” are small synthetic molecules isolated from combinatorial chemical libraries that will act as either agonists or antagonists to the targeted chemosensory proteins. Although they act in the same chemosensory and/or behavioral pathways as native pheromones, kairomones, or allomones, Arometics are not the native pheromone, kairomone, or allomone.

To develop Arometics, the chemosensory protein controlling a specific behavior like mating or feeding is identified using one of the methods common in the art, including but not limited to bioinformatic analysis of DNA sequences, amino acid sequences, or protein tertiary structure. Once the gene of this chemosensory protein has been isolated, it can be expressed in a heterologous system, such as E. coli. Combinatorial chemical libraries are screened for compounds capable of interacting using various in vitro or in vivo assays. Methods suitable for screening combinatorial chemical libraries for compounds capable of interacting with a chemosensory protein of interest include assay systems based on immobilizing the protein of interest on a surface monitored by a plasmon resonance biosensor and detecting interactions between the protein and a small molecule based on mass changes (this is the Deligo assay system), or using a fluorescent dye bound to the protein of interest and detecting fluorescence quenching when a small molecule displaces the dye on the protein's surface (the Attenu assay system).

The compounds identified by these screening systems as interacting with chemosensory proteins are then incorporated into products capable of altering pest species behavior based on their scent.

### VII. METHODS FOR IDENTIFYING COMPOUNDS THAT BIND CELL MEMBRANE RECEPTORS IN VIVO

Membrane-bound receptors such as GPCRs (also known as seven-transmembrane receptors or serpentine receptors) are an important component of many chemosensory processes, as they mediate signal transduction from the extracellular to the intracellular components responsible for detecting an external stimulus from the environment, be that a scent, odor, taste, or other chemical stimulus.¹ The invention provides means of identifying synthetic molecules, naturally occurring molecules, and other compounds such as can be found in combinatorial and/or natural chemical libraries for the ability to interact with the GPCR in vivo.

The invention concerns altering an organism's behavior by understanding and manipulating the chemosensory system at the molecular level. Therefore, an organism expressing the GPCR of interest can be placed in an established behavioral assay system common in the art—for example, insects are commonly evaluated in wind tunnel tests and/or Y-tube²⁵ tests, where the organism is presented with a choice of a chemical stimulus or a control—and numerous compounds can be screened in order to identify those compounds that affect the chemosensory pathway the GPCR of interest is involved in. This is accomplished by correlating the chemosensory pathway involving said GPCR with a behavior or response associated with it, and observing the organism to determine which compound(s) or molecule(s) can generate that behavioral response.

In the case of insects and other species that use antennae or similar structures as chemosensory organs, the in vivo assay described here can be performed without the entire organism by dissecting the antennae or chemosensory organs and using electroantennograms¹² to detect interactions between the GPCR of interest, which is expressed in the neuronal cells of the antennae, and the test molecules or compounds.

This method is applicable to organisms expressing the GPCR of interest natively, that is, as a result of their native genetic makeup, and it is also applicable in the case of an organism that is transformed to express a GPCR or other receptor of interest as a trans-gene. Thus, the invention provides the means to use a transgenic organism expressing a chemosensory receptor of interest in order to identify molecules or compounds that interact with that receptor.

Small molecules or compounds identified in this manner as interacting with a chemosensory protein of interest are called Arometics and can subsequently be evaluated for behavioral effects on living organisms.

### VIII. METHODS FOR IDENTIFYING COMPOUNDS THAT BIND TO OBPs AND/OR ARE OBP AGONISTS OR ANTAGONISTS IN VIVO

Odorant binding proteins (OBPs) and the similar gustatory binding proteins (GBPs; for the sake of this discussion, both classes will be called OBPs as they are essentially the same proteins) are an important component of many chemosensory processes, as they are among the first protein components expressed by an organism that interact with an external stimulus from the environment, be that a scent, odor, taste, or other chemical stimulus.^{1,3,9} The invention provides means of identifying synthetic molecules, naturally occurring molecules, and other compounds such as can be found in combinatorial and/or natural chemical libraries for the ability to interact with the OBP in vivo.

The invention concerns altering an organism's behavior by understanding and manipulating the chemosensory system at the molecular level. Therefore, an organism expressing the OBP of interest can be placed in an established behavioral assay system common in the art—for example, insects are commonly evaluated in wind tunnel tests and/or Y-tube²⁵ tests, where the organism is presented with a choice of a chemical stimulus or a control—and numerous compounds can be screened in order to identify those compounds that affect the chemosensory pathway the OBP of interest is involved in. This is accomplished by correlating the chemosensory pathway involving said OBP with a behavior or response associated with it, and observing the organism to determine which compound(s) or molecule(s) can generate that behavioral response.

In the case of insects and other species that use antennae or similar structures as chemosensory organs,¹ the in vivo assay described here can be performed without the entire organism by dissecting the antennae or chemosensory organs and using electroantennograms¹² to detect interactions between the OBP of interest, which is expressed in the neuronal cells of the antennae, and the test molecules or compounds.

This method is applicable to organisms expressing the OBP of interest natively, that is, as a result of their native genetic makeup, and it is also applicable in the case of an organism that is transformed to express a OBP or other chemosensory protein of interest as a trans-gene. Thus, the invention provides the means to use a transgenic organism expressing an OBP of interest in order to identify molecules or compounds that interact with that OBP. Small molecules or compounds identified in this manner as interacting with an OBP of interest are called Arometics and can subsequently be evaluated for behavioral effects on living organisms.

### IX. METHODS FOR IDENTIFYING COMPOUNDS THAT BIND TO CHEMOSENSORY PROTEINS AND/OR ARE CHEMOSENSORY PROTEIN AGONISTS OR ANTAGONISTS IN VIVO

Chemosensory proteins, including sensory appendage proteins (SAPs), odorant degrading enzymes (ODEs), circadian rhythm proteins and orthologs of the Drosophila melanogaster Takeout protein, pheromone binding proteins, and other proteins of the chemosensory system are key components of the molecular signaling cascade responsible for allowing an organism to detect and respond behaviorally to stimuli in its environment.^{1,3,9,11,16,20} The invention provides means of identifying synthetic molecules, naturally occurring molecules, and other compounds such as can be found in combinatorial and/or natural chemical libraries for the ability to interact with the chemosensory protein in vivo.

The invention concerns altering an organism's behavior by understanding and manipulating the chemosensory system at the molecular level. Therefore, an organism expressing the chemosensory protein of interest can be placed in an established behavioral assay system common in the art—for example, insects are commonly evaluated in wind tunnel tests and/or Y-tube²⁵ tests, where the organism is presented with a choice of a chemical stimulus or a control—and numerous compounds can be screened in order to identify those compounds that affect the chemosensory pathway the chemosensory protein of interest is involved in. This is accomplished by correlating the chemosensory pathway involving said chemosensory protein with a behavior or response associated with it, and observing the organism to determine which compound(s) or molecule(s) can generate that behavioral response.

In the case of insects and other species that use antennae or similar structures as chemosensory organs, the in vivo assay described here can be performed without the entire organism by dissecting the antennae or chemosensory organs and using electroantennograms¹² to detect interactions between the chemosensory protein of interest, which is expressed in the neuronal cells of the antennae, and the test molecules or compounds.

This method is applicable to organisms expressing the chemosensory protein of interest natively, that is, as a result of their native genetic makeup, and it is also applicable in the case of an organism that is transformed to express a chemosensory or other receptor of interest as a trans-gene. Thus, the invention provides the means to use a transgenic organism expressing a chemosensory protein of interest in order to identify molecules or compounds that interact with that receptor. Small molecules or compounds identified in this manner as interacting with a chemosensory protein of interest are called Arometics and can subsequently be evaluated for behavioral effects on living organisms.

### X. METHODS TO DEVELOP INSECT TRAPS UTILIZING COMPOUNDS CAPABLE OF ATTRACTING A TARGET SPECIES

Harmful mosquitoes such as Anopheles and Culex use olfactory stimuli as a means of identifying potential blood meal hosts.^{1,3,9,13,14,26,27} Consequently, devices that take advantage of the mosquitoes' sensitivity to odors can control pests by redirecting them away from humans and into traps or lures. The present invention recognizes this need and provides means, methods, and constructs to develop devices capable of emitting odors that mimic those odors commonly used to locate human hosts. The goal of these devices is to lure pests so that they can be trapped and/or subsequently eliminated using conventional insecticides within the trap itself or other means, such as an electrically charged grid.

OBPs or GPCRs from the target species can be isolated as described above and expressed in vitro, using tools common in the art that include transgenic eukaryotic cell lines or transgenic animals. Combinatorial chemical libraries are screened for compounds capable of interacting with the OBPs or GPCRs in vitro or in vivo, and these compounds are then further analyzed to determine which chemical structure(s) yield highly effective OBP or GPCR agonists. Behavioral assays utilizing live animals (for example, wind tunnel tests or other assays commonly used to study insect behavior) can be utilized to classify the compounds according to their effect on the target organism, and to identify attractants. These compounds will efficiently attract the targeted pest species, and can be incorporated into products capable of altering pest species behavior based on their scent. Such products include but are not limited to traps that selectively attract and kill mosquitoes by incorporating the isolated agonist and a highly toxic pesticide or an electrical grid.

These methods can also be employed to target other chemosensory proteins instead of or in addition to OBPs and GPCRs; such proteins include sensory appendage proteins (SAPs), odorant degrading enzymes (ODEs), orthologs of the Drosophila melanogaster Takeout protein (TOLs, for Takeout-likes), odorant receptors (ORs), gustatory receptors (GRs), pheromone binding proteins, other sensory proteins, circadian rhythm proteins and other proteins involved in olfaction, gustation, chemosensation, the sensory system, or the regulation of chemosensory-mediated behavior.^{1,3,9-11,16} Molecules or compounds identified in this manner as interacting with a chemo sensory protein of interest are called Arometics and can subsequently be evaluated for behavioral effects on living organisms.

### XI. METHODS TO DEVELOP INSECT REPELLENTS BASED ON AROMETICS ACTING AS SYNTHETIC AGONISTS OR ANTAGONISTS

Arometics, described previously herein, can be agonists and antagonists of OBPs, GPCRs, or other chemosensory proteins. Arometics can be utilized to manipulate any odor-based behavior provided the specific Arometic employed can interact with the chemosensory protein in the pathway controlling that behavior. If the behavior to be manipulated results in the insect being repelled, Arometics can be used to develop novel insect repellents. Arometics can also results in the inability of an insect to detect a given semiochemical, that is, in anosmia or agustia. These Arometics are not strictly repellents, as they do not actively repel the insect; however, the insect is no longer attracted to the chemical in question, resulting in altered behavior. Chemosensory proteins from the target species are isolated and characterized as described above; combinatorial chemical libraries are screened for compounds capable of interacting with the chemosensory proteins in vitro or in vivo, and these compounds are then further analyzed to determine which chemical structure(s) yield highly effective chemosensory protein agonists. Behavioral assays utilizing live animals (for example, wind tunnel tests or other assays commonly used to study insect behavior) can be utilized to classify the compounds according to their effect on the target organism, and to identify repellents or compound that induce anosmia or agustia. Provided the chemosensory protein in question is part of a pathway that causes the insect to avoid a specific scent, these compounds will efficiently repel the targeted pest species. The Arometics can be incorporated into products capable of repelling pest species based on their scent.

These methods can also be employed to generate Arometics that act as synthetic agonists or antagonists by targeting other chemosensory proteins instead of or in addition to OBPs; such proteins include sensory appendage proteins (SAPs), odorant degrading enzymes (ODEs), orthologs of the Drosophila melanogaster Takeout protein (TOLs, for Takeout-likes), odorant receptors (ORs), gustatory receptors (GRs) and other proteins involved in olfaction, gustation, chemosensation, or the regulation of chemosensory-mediated behavior.^{1,3,9-11,16}

Arometics devised in this manner can be delivered in a variety of mechanisms including gels, emulsions, sprays, slow-release capsules, suspensions, solutions, volatile solids, liquids, and gases. The Arometics can be included in fabrics or materials used for bed nets, protective netting, or other garments.

### EXAMPLES

### Example 1

### Identifying Compounds or Molecules that Interact with an Invertebrate Chemosensory Protein and Using Those Compounds as the Basis for Developing an Arometic—a Novel Control Product

The mosquito, Culex pipiens, poses as public health risk to human, equine, avian, and other populations as it is a vector for the West Nile virus.²⁸ Culex uses chemosensory cues in order to locate its prey, locate a mate, and determine a location to lay eggs.²⁹ Thus, a method of isolating compounds that interact (bind to) specific protein components of this mosquito's chemosensory system is beneficial in the development of novel control products that operate by altering the chemosensory apparatus' functionality. Proteins of interest include OBPs, sensory appendage proteins (SAPs), odorant degrading enzymes (ODEs), orthologs of the Drosophila melanogaster Takeout protein (TOLs, for Takeout-likes), odorant receptors (ORs), gustatory receptors (GRs) and other proteins involved in olfaction, gustation, chemosensation, or the regulation of chemosensory-mediated behavior.^{1,3,9-11,16}

For example, several OBPs, including OBP9, OBP20 and OBP48, are enriched in the antennae and heads of the mosquito species Anopheles gambiae,^{9,27} making Culex pipiens orthologs potentially interesting chemosensory proteins to target in this assay system. The gene encoding these orthologs can be isolated by screening an antennal-specific cDNA library for transcripts encoding these proteins based on the known sequence of orthologs from other species, including Anopheles gambiae. A suitable cDNA transcript can then be cloned into an expression vector in order to generate recombinant Culex pipiens OBP protein (CpipOBP) as is common in the art.

The Deligo assay system is then used to identify binding partners for the chemosensory protein of interest. Recombinant CpipOBP protein is immobilized covalently onto a surface suitable for use in a plasmon resonance biosensor such as those commonly available by firms including Biacore AG (Sweden) or Reickert Analytical Instruments (United States). An aqueous buffer is allowed to flow over the CpipOBP surface, and samples of small molecules, proteins, compounds, or other potential binding partners are introduced. A binding event between CpipOBP and another molecule in this assay system results in a net mass increase on the surface containing the protein and its binding partner, and this net mass change can be detected optically due to the surface plasmon resonance effect. Potential binding partners can be derived from combinatorial chemical libraries, natural product libraries, collections of known pharmacophores, collections of proteins, and other compound collections.

Substances or molecules that bind the chemosensory protein tested—in this case, CpipOBP—have the potential to affect the chemosensory pathway that protein is involved in and thus alter the way in which the target organism responds to external stimuli or behaves. These substances are thus lead compound that can be refined chemically as needed to develop Arometics, discussed elsewhere.

Instead of surface plasmon resonance, flow cytometry can be used to detect the interaction between CpipOBP and a binding partner. A dye, such as 1-NPN, will fluoresce when captured by the ligand-binding pocket of a chemosensory protein and this fluorescence is quenched when the dye is displaced by another molecule. Therefore, flow cytometry can be used to detect fluorescence quenching and thus isolate novel binding partners for CpipOBP from the classes of molecules listed above.

Another approach for detecting novel interactors uses the same principle of detecting the fluorescence quenching once a dye such as 1-NPN is displaced from the binding pocket of a chemosensory protein, but multiplexes the detection step by using a multiwell plate with each well containing a protein:dye solution. These wells can each be tested with a different potential binding partner for the chemosensory protein, and the fluorescence quenching that results when a binding partner displaces the dye from the protein can be detected using a tuneable (variable wavelength) spectrofluorometer such as that commonly available by firms including Molecular Devices (United States). This is the Attenu assay system.

### Example 2

### Identifying Compounds or Molecules that Interact with an Invertebrate Chemosensory Protein by Using a Cell-Based Assay System that Enhances the Sensitivity of a GPCR-Based Binding Assay

Binding assays to detect compounds or molecules that bind to GPCRs, serpentine receptors, seven-transmembrane receptors, or other membrane-bound receptors are common in the field of drug discovery. Existing techniques in question utilize a cell-based assay system, where a GPCR of interest is expressed on the cell membrane and any binding event on the GPCR's surface triggers a detectable reaction. This invention improves significantly upon existing techniques by utilizing a soluble chemosensory protein from the class of proteins that escort external stimuli, which are typically hydrophobic, through the hydrophilic extracellular medium or haemolymph to the GPCR on the neuronal cell surface in vivo. Such soluble proteins are OBPs, SAPs, or other soluble effectors.¹ Recent data indicate that these proteins form complexes with the external chemical stimulus molecule, and that these complexes have improved binding to the GPCR.²⁴ This invention therefore utilizes OBPs or SAPs in conjunction with GPCRs in cell-based assays, resulting in more efficient and more sensitive assay systems.

For example, the Anopheles gambiae OBP48^{3,9,27} is a potentially interesting chemosensory protein to use in this assay system for the reasons discussed previously. The gene encoding OBP48 can be isolated by screening an antennal-specific cDNA library for transcripts encoding the OBP48 protein. A suitable cDNA transcript can then be cloned into an expression vector in order to generate recombinant Anopheles gambiae OBP48 protein as is common in the art.

Recombinant OBP48 is them introduced into a cell-based assay system that tests the GPCR associated with the chemosensory pathway of interest for the ability to bind a series of candidate molecules, compounds, or substances. The presence of recombinant OBP48 enhances the sensitivity of the assay system and allows better detection of potential molecular interactors.

Substances or molecules that complex with the chemosensory protein utilized—in this case, OBP48—and then activate the given GPCR have the potential to affect the chemosensory pathway involved and thus alter the way in which the target organism responds to external stimuli or behaves. These substances are thus lead compound that can be refined chemically as needed to develop Arometics, discussed elsewhere.

### Example 3

### Developing a Control Product for the Mosquito Anopheles Gambiae by Identifying Chemosensory Protein Agonists or Antagonists

Arometics are novel effectors derived from combinatorial chemical libraries, natural compound libraries, or other molecule collections. Arometics can be identified by subjecting a chemosensory protein expressed in vitro to a screening process with the Deligo or Attenu assay systems, detailed elsewhere in this document. Arometics can be agonists or antagonists of chemosensory proteins such as OBPs or GPCRs, and can thus manipulate odor-based behavior in the targeted species. Other chemosensory proteins that can be targeted for the development of Arometics include sensory appendage proteins (SAPs), odorant degrading enzymes (ODEs), orthologs of the Drosophila melanogaster Takeout protein (TOLs, for Takeout-likes), odorant receptors (ORs), gustatory receptors (GRs) and other proteins involved in olfaction, gustation, chemosensation, or the regulation of chemosensory-mediated behavior or circadian rhythms, feeding, and mating.^{1,3,9-11,16}

The mosquito, Anopheles gambiae, expresses a number of chemosensory proteins that are regulated according to life cycle stage and specific chemosensory or behavioral response.^{1,13,14,26,30,31} Orthologs of the Drosophila melanogaster Takeout protein (TO) are one such chemosensory protein type expressed in A. gambiae.^{1,3,9-11,16,27} TO regulates circadian rhythms, and may therefore be important in the regulation of nighttime feeding behavior in A. gambiae. Mosquitoes expressing TO can be exposed to a series of test compounds and assayed for behavioral responses in order to determine which compounds interact with the OBP in question; these compounds can then be classified as agonists or antagonists biochemically in vitro using techniques common in the art.

TO or other chemosensory proteins from Anopheles gambiae can be transformed into another species, such as Drosophila melanogaster, and that transgenic organism can be subjected to the same behavioral assays described here in order to identify chemosensory protein agonists or antagonists.

The compounds identified alter Anopheles behavior and are lead compounds for the development of Arometics. Arometics devised in this manner can be delivered in a variety of mechanisms including gels, emulsions, sprays, slow-release capsules, suspensions, solutions, volatile solids, liquids, and gases. The Arometics can be included in fabrics or materials used for bed nets, protective netting, or other garments.

### Example 4

### Developing a Novel Insect Control Product that Operates by Reducing a Target Species' Sensitivity to a Specific Odor, Taste, or Other Chemosensory Stimulus Using the Honeybee, Apis Mellifera, as a Model System

The honeybee, Apis mellifera, is an economically significant insect species responsible for the majority of insect-mediated pollination of plant cultivars. However, it is also considered a pest in domestic situations and poses a health risk to sensitive humans or when present in the Africanized state.³²⁻³⁶ Bees have a wealthy chemosensory “language” comprising scents and odors such as pheromones, allomones, synomones, and kairomones,³⁷⁻⁴⁴ and this invention provides the methods to manipulate honeybee behavior by reducing the bee's sensitivity to the components of this chemosensory language.

The honeybee protein, ASP1, is a specialized OBP called a pheromone binding protein or PBP that binds to queen pheromone in vivo.⁴² ASP2 is an OBP that binds a number of tested scents or odors.⁴¹ Both these proteins are examples of proteins that can be targeted in this assay. Other potential target proteins are sensory appendage proteins (SAPs), odorant degrading enzymes (ODEs), orthologs of the Drosophila melanogaster Takeout protein (TOLs), other pheromone binding proteins, other OBPs, circadian rhythm proteins and other proteins involved in olfaction, gustation, chemosensation, the sensory system, or the regulation of chemosensory-mediated behavior.^{1,3,9-11,16}
The target chemosensory protein is involved in a pathway resulting in a particular behavior in the presence of the natural stimulus for that pathway. Bees expressing the chosen target protein are exposed to the natural stimulus and their response in the presence of test compound vs. the absence of test compound is assayed. Test compounds can be sourced from a combinatorial chemical library, a natural product library, or other molecule collection; in order to identify candidate test compounds from these collections, the Attenu or Deligo assays systems (detailed elsewhere in this document) can be used. Behavioral assays, wind tunnel tests, and Y-tube²⁵ tests are all examples of in vivo tests using living bees that are applicable. The assay is designed to detect altered, reduced behavioral response to the natural scent, odor, or stimulus in the presence of the test compound. Furthermore, the assay can be performed using electroantennograms¹² to detect any reduction in response of dissected bee antennae to the natural stimulus in the presence of a tested compound.
The sensitivity of the bee to the natural stimulus can be reduced to the point of anosmia, (total inability to smell) or agustia (total inability to taste). Compounds capable of reducing the sensitivity of the chemosensory system or of inducing anosmia or agustia are Arometics and can be used as the basis for novel insect control strategies.

### Example 5

### A Novel Mating Disruption Method to Control the Codling Moth, Cydia Pomonella

The codling moth is a significant pest of stone fruit in the United States.^{45,46} This species has a known sensitivity to pheromones and kairomones as well as synthetic odors that have been used in lures and bait stations.^{45,47-51} In this example an Arometic is developed for use in lures and traps for the codling moth.

Codling moths respond to kairomones emitted by fruit trees such as apple trees. Shotgun sequencing of antennal-specific cDNA libraries, bioinformatic analysis, or other means common in the art can be used to isolate codling moth OBPs involved in recognizing these kairomones, and thus in attracting codling moths to apple trees. One such OBP can be selected as a target for the development of a novel, Arometics-based mating disruption strategy for the codling moth; this novel method is based on inducing anosmia, or the inability to detect the chemosensory molecules (pheromones or other scents) responsible for the mating response behavior (in contrast, traditional mating disruption techniques are based on hyperstimulating the chemosensory pathway responsible for the mating behavior, and are thus more accurately viewed as mating confusion approaches).

One OBP of interest is the pheromone binding protein (PBP) potentially involved in mating behavior, called PBP1. For this example, PBP1 is expressed in vitro as a recombinant fusion protein using common methods and is evaluated using one of the techniques presented previously; PBP1 can be evaluated using surface plasmon resonance, flow cytometry, a cell-based assay system, or a fluorescence-based assay system (see the Deligo and Attenu assay systems in this document). The goal of these tests is to identify molecules or compounds capable of binding to or interacting with the OBP at the molecular level; candidate compounds are sourced from combinatorial chemical libraries, protein libraries, natural compound libraries, or other sources.

Compounds or molecules identified as binding partners for the specific OBP tested are then evaluated in behavioral assays to determine if they alter the insect's behavior in vivo; specifically they are tested for attractant qualities. The target OBP is involved in a pathway resulting in a particular behavior in the presence of the natural stimulus for that pathway—in this case, attraction of the moth to the fruit tree. Behavioral assays, wind tunnel tests, and Y-tube²⁵ tests are all examples of in vivo tests using living moths that are applicable. Furthermore, the assay can be performed using electroantennograms¹² to detect any reduction in response of dissected bee antennae to the natural stimulus in the presence of a tested compound.

Attractant Arometics identified in this manner can be encapsulated in time-release gels, liquids, suspensions, mixtures, aerosols, or other formulas and incorporated into traps or lures. The lures can simple trap the moths, or they can incorporate a poison to kill the moths as well.

### Example 6

### Another Novel Trap for the Codling Moth, Cydia Pomonella; Taste-Based Lure

The codling moth is a significant pest of stone fruit in the United States^{45,46} and the adult moths are nectar-feeders. In this example an Arometic based on stimulating an adult moth's sense of taste is developed for use in lures and traps for the codling moth.

Codling moths respond to the taste of nectar in flower blossoms. Shotgun sequencing of antennal-specific cDNA libraries, bioinformatic analysis, or other means common in the art can be used to isolate codling moth chemosensory proteins involved in recognizing these tastes, and thus in attracting codling moths to apple trees. One such chemosensory protein can be selected as a target for the development of a novel, Arometics-based trap for the codling moth.

Once identified, the chemosensory protein targeted is expressed in vitro as a recombinant fusion protein using common methods and is evaluated using one of the techniques presented previously; for example, the chemosensory protein can be evaluated using surface plasmon resonance, flow cytometry, a cell-based assay system, or a fluorescence-based assay system. The goal of these tests is to identify molecules or compounds capable of binding to or interacting with the chemosensory protein at the molecular level; candidate compounds are sourced from combinatorial chemical libraries, protein libraries, natural compound libraries, or other sources.

Compounds or molecules identified as binding partners for the specific chemosensory protein tested are then evaluated in behavioral assays to determine if they alter the insect's behavior in vivo; specifically they are tested for attractant qualities. Live moths are fed the test compound during the behavioral assay. Arometics identified as attractive to moths in this manner can be encapsulated in time-release gels, liquids, suspensions, mixtures, aerosols, or other formulas and incorporated into traps or lures. The lures can simple trap the moths, or they can incorporate a poison to kill the moths as well.

### Example 7

### Identification, Cloning, and Characterization of Anopheles Gambiae Odorant-Binding Proteins. Developing an OBP-Binding Compound Capable of Altering Anopheles Behavior by Inhibiting OBP-Binding Protein Function. Developing a Novel Form of Insect Repellent

Anopheles gambiae mosquitoes are relatively widespread and are known to harbor the infectious agent responsible for malaria in humans. In general, previous efforts to control Anopheles populations have been based on toxic pesticides. The present invention provides the compositions and methods desirable to clone and characterize odorant-binding proteins responsible for efficient olfaction in Anopheles. Since this species relies on olfactory information to find mates and humans on which to feed, the present invention provides compositions and methods to develop non-toxic pest control products. For example, characterizing Anopheles OBPs could provide the information desirable to develop a pest control approach based on mating disruption or another form of behavior alteration, since successful mating depends on the male Anopheles locating a female ready to mate. Another feasible approach using the compositions and methods provided by the present invention is to develop a pest control approach based on rendering Anopheles incapable of detecting the scent of humans. In this manner, products developed based on the compositions and methods provided by the present invention will replace current insect repellents.

The present invention provides methods of cloning genes encoding odorant-binding proteins and subsequently identifying compounds or chemicals capable of binding the OBPs in order to block their normal function. By inducing anosmia, these compounds can be used to alter pest behavior by prohibiting members of one gender to find members of the other gender, eliminating the mating response entirely, or prohibiting pests from locating humans on which to feed.

Several thousand Anopheles mosquitoes are separated into male and female genders. The antennae of each gender are dissected separately, and mRNA is isolated from each pool of antennae. MRNA is also extracted from the bodies of both genders. cDNA is transcribed from each mRNA sample. One portion of the extracted cDNA is reserved in order to make labeled hybridization probes later, while the rest is used to construct cDNA libraries in a suitable vector; examples include but are not limited to bacterial plasmids or .lambda. phage. This process yields three cDNA libraries; one library contains clones representing the transcripts from male antennae, another library contains clones from mRNA representing the transcripts from female antennae, and a third library contains clones representing the mRNA transcripts from the mosquito bodies.

The libraries with clones from the antennae of both genders are arrayed on nitrocellulose filters and screened with a labeled probe made from cDNA expressed in the bodies. Positive clones are discarded in order to examine only those clones specific to the antennae, since the genes encoding OBPs will be expressed selectively in antennal tissues rather than elsewhere in the body. The female antennal library is screened with a probe derived from female antennal cDNA and negative clones are eliminated or reduced because they lack an insert. Highly expressed clones are sequenced and the data analyzed for the presence of cDNAs encoding OBPs. Approximately 15% of all antennal cDNAs were found to encode OBPs.

The present invention provides compositions and methods for functional analysis of an OBP and for identifying compounds capable of binding an OBP optionally with high affinity. Combinatorial chemical libraries, either commercially available or constructed in a proprietary manner, are screened to identify compounds capable of binding OBPs expressed in vitro, and these lead compounds are evaluated in behavioral assays to determine whether they are capable of blocking normal responses to odors and pheromones in Anopheles adults.

These procedures will yield a compound capable of altering the mating and feeding behaviors of adult Anopheles mosquitoes. Such a compound can have a number of applications, including use as an agent in pest management programs implementing mating disruption or in repellent products designed to make humans undetectable or reduce detectability by mosquitoes searching for a blood meal. Furthermore, such a compound can have a relatively simple chemical composition that lends itself to manipulation in order to attain desirable physical properties. For example, the compound's viscosity, pH, solubility, and other properties can be modified to make it more suitable for deployment in a range of environments, or as an agent in a variety of products. The compound may thus be used in bracelets or necklaces, or in bed netting, fabrics, powders, gels, liquids, or emulsions.

### Example 8

### Developing Silverfish and Firebrat Repellents that can be Incorporated into Solids in Order to Control Pests such as Lepisma Saccharina and Thermobia Domestica

The common silverfish, Lepisma saccharina, and the firebrat, Thermobia domestica, are similarly shaped, relatively primitive insect pests of the order Thysanura that consume and/or masticate material high in protein, sugar, or starch. Their target foods can include cereals, flour, books, paper, glue, wallpaper, cotton, linen, silk, rayon, and paste, making them a significant domestic, agricultural, and commercial nuisance.

The present invention recognizes the need to develop effective repellents against these pests, and provides the means and compositions desirable to isolate and identify compounds with desirable effects and chemical or physical properties to be incorporated into these repellents. Desirable properties include but are not limited to the ability to be integrated in packing materials (paper, cardboard, plastics, or fabrics) and building materials.

Repellents are composed of molecules or compounds isolated from combinatorial chemical libraries based on their ability to interact with odorant proteins (for example, OBPs and GPCRs) from Lepisma or Thermobia using the means and methods of the present invention. Briefly, sensory tissue (antennae) are dissected and tissue-specific cDNA libraries of clones representing the genes expressed in antennal tissue are constructed using the techniques described herein. These libraries are screened for clones encoding OBPs or GPCRs, using methods described herein; the genes identified are expressed in vitro and combinatorial chemical libraries are screened for compounds that interact with the OBP or GPCR in question. Compounds capable of activating olfactory pathways controlling aversive reactions or compounds capable of inducing anosmia can be incorporated into repellents; the effect of such compounds can be verified by conducting electrophysiological experiments, including electroantennograms, or behavioral assays using either Lepisma or Thermobia specimens, using methods described herein. Thus, the present invention provides the means and compositions to develop a pest repellent incorporated into cardboard, fabric, or paper to protect stored foods, or a repellent to protect paper products, building materials, structural materials, fabrics, and storage materials.

### Example 9

### Protecting a Vineyard from Infection by the Glassy-Winged Sharpshooter, Homalodisca Coagulata, Using Novel Repellents or Attractants

The Glassy-winged sharpshooter, Homalodisca coagulata, poses a serious threat to citrus and vineyards in California and elsewhere. The present invention recognizes the need to develop novel, highly effective products to control this insect pest, and provides methods and compositions to do so. These products can generally be classified as either repellents or attractants; both classes of product are based on compounds isolated from combinatorial chemical libraries based on their ability to interact with insect olfactory proteins, including OBPs and GPCRs. Thus, the initial steps involving screening combinatorial chemical libraries for compounds capable of interacting with olfactory proteins from Homalodisca are common to the development of either a repellent or an attractant. A brief example of each implementation follows.

Repellents: Compounds capable of activating olfactory pathways controlling aversive reactions or compounds capable of inducing anosmia can be incorporated into repellents. These repellents can be deployed as aerosols, gels, or sprays to coat vines or plants, or as powders or solids. These repellents will prevent the insects from locating food or mates, or generate a distasteful odor to drive the insects away from the fields being protected.

Attractants: Compounds capable of attracting either or both sexes of Homalodisca can be incorporated into attractants. These attractants can be used to construct traps or lures in a bait-and-kill pest control scheme, where the insects are attracted to a toxin, subsequently kills them, or a trap that immobilizes them.

### REFERENCES

All publications, including patent documents and scientific articles, referred to in this application and the bibliography and attachments are incorporated herein by reference in their entirety for all purposes to the same extent as if each individual publication were individually incorporated by reference. All headings are for the convenience of the reader and should not be used to limit the meaning of the text that follows the heading, unless so specified.
1. Justice, R. W., Biessmann, H., Walter, M. F., Dimitratos, S. D. & Woods, D. F. Genomics spawns novel approaches to mosquito control. Bioessays 25, 1011-20 (2003).
2. Carlson, J. R. Olfaction in Drosophila: from odor to behavior. Trends Genet 12, 175-80 (1996).
3. Biessmann, H., Walter, M. F., Dimitratos, S. & Woods, D. Isolation of cDNA clones encoding putative odourant binding proteins from the antennae of the malaria-transmitting mosquito, Anopheles gambiae. Insect Mol Biol 11, 123-32 (2002).
4. Kim, M. S., Repp, A. & Smith, D. P. LUSH odorant-binding protein mediates chemosensory responses to alcohols in Drosophila melanogaster. Genetics 150, 711-21 (1998).
5. Kim, M. S. & Smith, D. P. The invertebrate odorant-binding protein LUSH is required for normal olfactory behavior in Drosophila. Chem Senses 26, 195-9 (2001).
6. Firestein, S. Olfaction: scents and sensibility. Curr Biol 6, 666-7 (1996).
7. Keller, L. & Parker, J. D. Behavioral genetics: a gene for supersociality. Curr Biol 12, R180-1 (2002).
8. Krieger, M. J. & Ross, K. G. Identification of a major gene regulating complex social behavior. Science 295, 328-32 (2002).
9. Justice, R. W., Dimitratos, S., Walter, M. F., Woods, D. F. & Biessmann, H. Sexual dimorphic expression of putative antennal carrier protein genes in the malaria vector Anopheles gambiae. Insect Mol Biol 12, 581-94 (2003).
10. Dauwalder, B., Tsujimoto, S., Moss, J. & Mattox, W. The Drosophila takeout gene is regulated by the somatic sex-determination pathway and affects male courtship behavior. Genes Dev 16, 2879-92 (2002).
11. So, W. V. et al. takeout, a novel Drosophila gene under circadian clock transcriptional regulation. Mol Cell Biol 20, 6935-44 (2000).
12. Park, K. C., Ochieng, S. A., Zhu, J. & Baker, T. C. Odor Discrimination using Insect Electroantennogram Responses from an Insect Antennal Array. Chem Senses 27, 343-52 (2002).
13. Costantini, C., Sagnon, N., della Torre, A. & Coluzzi, M. Mosquito behavioural aspects of vector-human interactions in the Anopheles gambiae complex. Parassitologia 41, 209-17 (1999).
14. Hallem, E. A., Nicole Fox, A., Zwiebel, L. J. & Carlson, J. R. Olfaction: mosquito receptor for human-sweat odorant. Nature 427, 212-3 (2004).
15. Vogt, R. G., Rogers, M. E., Franco, M. D. & Sun, M. A comparative study of odorant binding protein genes: differential expression of the PBP1-GOBP2 gene cluster in Manduca sexta (Lepidoptera) and the organization of OBP genes in Drosophila melanogaster (Diptera). J Exp Biol 205, 719-44 (2002).
16. Sarov-Blat, L., So, W. V., Liu, L. & Rosbash, M. The Drosophila takeout gene is a novel molecular link between circadian rhythms and feeding behavior. Cell 101, 647-56 (2000).
17. Galindo, K. & Smith, D. P. A large family of divergent Drosophila odorant-binding proteins expressed in gustatory and olfactory sensilla. Genetics 159, 1059-72 (2001).
18. Hill, C. A. et al. G protein-coupled receptors in Anopheles gambiae. Science 298, 176-8 (2002).
19. Shanbhag, S. R., Park, S. K., Pikielny, C. W. & Steinbrecht, R. A. Gustatory organs of Drosophila melanogaster: fine structure and expression of the putative odorant-binding protein PBPRP2. Cell Tissue Res 304, 423-37 (2001).
20. Bohbot, J. & Vogt, R. G. Antennal expressed genes of the yellow fever mosquito (Aedes aegypti L.); characterization of odorant-binding protein 10 and takeout. Insect Biochem Mol Biol 35, 961-79 (2005).
21. Ban, L. et al. Biochemical characterization and bacterial expression of an odorant-binding protein from Locusta migratoria. Cell Mol Life Sci 60, 390-400 (2003).
22. Ban, L., Zhang, L., Yan, Y. & Pelosi, P. Binding properties of a locust's chemosensory protein. Biochem Biophys Res Commun 293, 50-4 (2002).
23. Jin, X. et al. Expression and immunolocalisation of odorant-binding and chemosensory proteins in locusts. Cell Mol Life Sci 62, 1156-66 (2005).
24. Kaissling, K. E. Olfactory perireceptor and receptor events in moths: a kinetic model. Chem Senses 26, 125-50 (2001).
25. Knight, A. L. & Light, D. M. Attractants from Bartlett pear for codling moth, Cydia pomonella (L.), larvae. Naturwissenschaften 88, 339-42 (2001).
26. Dekker, T., Steib, B., Carde, R. T. & Geier, M. L-lactic acid: a human-signifying host cue for the anthropophilic mosquito Anopheles gambiae. Med. Vet. Entomol. 16, 91-98 (2002).
27. Biessmann, H., Nguyen, Q. K., Le, D. & Walter, M. F. Microarray-based survey of a subset of putative olfactory genes in the mosquito Anopheles gambiae. Insect Mol Biol 14, 575-89 (2005).
28. Petersen, L. R. & Roehrig, J. T. West Nile virus: a reemerging global pathogen. Emerg Infect Dis 7, 611-4 (2001).
29. Braverman, Y., Kitron, U. & Killick-Kendrick, R. Attractiveness of vertebrate hosts to Culex pipiens (Diptera: Culicidae) and other mosquitoes in Israel. J Med Entomol 28, 133-8 (1991).
30. Fox, A. N., Pitts, R. J., Robertson, H. M., Carlson, J. R. & Zwiebel, L. J. Candidate odorant receptors from the malaria vector mosquito Anopheles gambiae and evidence of down-regulation in response to blood feeding. Proc Natl Acad Sci USA 98, 14693-7 (2001).
31. Nighorn, A. & Hildebrand, J. G. Dissecting the molecular mechanisms of olfaction in a malaria-vector mosquito. Proc Natl Acad Sci USA 99, 1113-4 (2002).
32. Stort, A. C. Genetic study of the aggressiveness of two subspecies of Apis mellifera in Brazil. IV. Number of stings in the gloves of the observer. Behav Genet 5, 269-74 (1975).
33. Schumacher, M. J., Schmidt, J. O., Egen, N. B. & Lowry, J. E. Quantity, analysis, and lethality of European and Africanized honey bee venoms. Am J Trop Med Hyg 43, 79-86 (1990).
34. McKenna, W. R. The Africanized honey bee. Allergy Proc 13, 7-10 (1992).
35. Tunget, C. L. & Clark, R. F. Invasion of the ‘killer’ bees. Separating fact from fiction. Postgrad Med 94, 92-4, 97-8, 101-2 (1993).
36. Kim, K. T. & Oguro, J. Update on the status of Africanized honey bees in the western states. West J Med 170, 220-2 (1999).
37. Smith, B. H. & Cobey, S. The olfactory memory of the honeybee Apis mellifera. II. Blocking between odorants in binary mixtures. J Exp Biol 195, 91-108 (1994).
38. Moore, P. A., Price, B. A. & Schneider, R. W. Antennal morphology as a physical filter of olfaction: temporal tuning of the antennae of the honeybee, Apis mellifera. J Insect Physiol 44, 677-684 (1998).
39. Gerber, B. & Smith, B. H. Visual modulation of olfactory learning in honeybees. J Exp Biol 201 (Pt 14), 2213-7 (1998).
40. Reinhard, J., Srinivasan, M. V. & Zhang, S. Olfaction: scent-triggered navigation in honeybees. Nature 427, 411 (2004).
41. Briand, L., Nespoulous, C., Huet, J. C., Takahashi, M. & Pernollet, J. C. Ligand binding and physico-chemical properties of ASP2, a recombinant odorant-binding protein from honeybee (Apis mellifera L.). Eur J Biochem 268, 752-60 (2001).
42. Briand, L., Nespoulous, C., Huet, J. C. & Pernollet, J. C. Disulfide pairing and secondary structure of ASP1, an olfactory-binding protein from honeybee (Apis mellifera L). J Pept Res 58, 540-5 (2001).
43. Wanner, K. W. et al. Analysis of the insect os-d-like gene family. J Chem Ecol 30, 889-911 (2004).
44. Calvello, M. et al. Expression of odorant-binding proteins and chemosensory proteins in some Hymenoptera. Insect Biochem Mol Biol 35, 297-307 (2005).
45. Knight, A. L. Monitoring codling moth (Lepidoptera: Tortricidae) with passive interception traps in sex pheromone-treated apple orchards. J Econ Entomol 93, 1744-51 (2000).
46. Caprile, J. L. et al. (University of California, 2002).
47. Backman, A. C. et al. Antennal response of codling moth males, Cydia pomonella L. (Lepidoptera: Tortricidae), to the geometric isomers of codlemone and codlemone acetate. J Comp Physiol [A] 186, 513-9 (2000).
48. Light, D. M. et al. A pear-derived kairomone with pheromonal potency that attracts male and female codling moth, Cydia pomonella (L.). Naturwissenschaften 88, 333-8 (2001).
49. Losel, P. M., Potting, R. P., Ebbinghaus, D. & Scherkenbeck, J. Factors affecting the field performance of an attracticide against the codling moth Cydia pomonella. Pest Manag Sci 58, 1029-37 (2002).
50. Tomaszewska, E. et al. Evaluation of pheromone release from commercial mating disruption dispensers. J Agric Food Chem 53, 2399-405 (2005).
51. Welter, S. et al. Pheromone mating disruption offers selective management options for key pests. California Agriculture 59, 17-22 (2005).

1-10. (canceled)

11) A method of identifying molecules that are OBP agonists or antagonists, comprising the steps of:
a) providing an animal expressing an OBP of interest;
b) providing a library of test compounds;
c) exposing said animal to at least one test compound; and
d) observing changes in said animal's behavior, whereby the OBP agonist or antagonist is identified.

12) A method of identifying molecules that are chemosensory protein agonists or antagonists, comprising the steps of:
a) providing an animal expressing the protein of interest;
b) providing a library of test compounds;
c) exposing said animal to at least one test compound; and
d) observing changes in said animal's behavior, whereby the protein agonist or antagonist is identified.

13) (canceled)

14) The method of claim 12, wherein said protein is a sensory appendage protein, soluble olfactory protein, ortholog of Drosophila Takeout protein, circadian rhythm protein, pheromone binding protein, or other soluble protein involved in the sensory system.

15) A method of reducing a target animal's sensitivity to odors, comprising the steps of:
a) providing a compound known to interact with OBPs of a target species;
b) incorporating said compound into products capable of altering pest species behavior; and
c) exposing said target animal to the product containing said compound.

16) A method of reducing a target animal's sensitivity to chemosensory cues, comprising:
a) providing a compound known to interact with chemosensory proteins of a target species;
b) incorporating said compound into products capable of altering pest species behavior; and
c) exposing said target animal to the product containing said compound.

17) The method of claim 16, wherein said chemosensory protein is a gustatory-binding protein, sensory appendage protein, soluble olfactory protein, ortholog of Drosophila Takeout protein, circadian rhythm protein, pheromone binding protein, gustatory receptor, or other protein involved in the sensory system.

18) A method of trapping invertebrates with attractants, comprising the steps of:
a) providing a compound known to interact with an OBP and [/or GPCR] of a target species;
b) incorporating said compound into a trap that will selectively attract said invertebrate; and
c) exposing said invertebrate to the trap, whereby said invertebrate is trapped.

19) The method of claim 18, wherein said trap further comprises a poison sufficient to kill said trapped invertebrate.

20) A method of trapping invertebrates with attractive tastes or other attractive chemosensory cues, comprising the steps of:
a) providing a compound known to interact with a chemosensory protein of a target species;
b) incorporating said compound into a trap that will selectively attract said invertebrate; and
c) exposing said invertebrate to the trap, whereby said invertebrate is trapped.

21) The method of claim 20, wherein said trap further comprises a poison sufficient to kill said trapped invertebrate.

22) The method of claim 20, wherein said chemosensory protein is a gustatory-binding protein, sensory appendage protein, soluble olfactory protein, ortholog of Drosophila Takeout protein, circadian rhythm protein, pheromone binding protein, gustatory receptor or other protein involved in the sensory system.

23) A method of repelling invertebrates with Arometics, comprising the steps of:
a) providing at least one Arometic;
b) incorporating said Arometic into a delivery system that will selectively repel said invertebrate; and
c) exposing said invertebrate to the delivery system, whereby said invertebrate is repelled.

24) The method of claim 23, wherein the delivery system is a gel, a spray, an emulsion, a slow-release capsule, or a liquid, solid, or gas included in fabrics or materials used for bed nets, other protective netting, or garments.

25) (canceled)
